(19) United States
(12) Patent Application Publication
(10) Pub. No.: US 2005/0244462 A1
(43) Pub. Date: Nov. 3, 2005

(54) Devices and methods for treating a mammalian eye

(75) Inventor: Sidiq Farooq, Newark, CA (US)

Correspondence Address: STOUT, UXA, BUYAN & MULLINS LLP, 4 VENTURE, SUITE 300, IRVINE, CA, 92618, US

(73) Assignee: Allergan, Inc., Irvine, CA (US)

(21) Appl. No.: 10/836,908

(22) Filed: Apr. 30, 2004

(51) Int. Cl. A61F 2/00

(52) U.S. Cl. 424/427

(57) ABSTRACT

Devices and methods for delivering a therapeutically active ocular implant into an eye are provided. Such a device generally includes a hollow sleeve having an open proximal end, a distal end, a substantially uniform inner diameter and a substantially uniform outer diameter extending between the proximal end and the open distal end. In addition, the device includes a plunger element having an angled configuration and sized to be slidably received within the sleeve for urging an implant from the distal end of the sleeve and into an eye. Systems for performing ocular research are provided which include a plurality of such hollow sleeves cut from a single flouroplastic tube, and a plurality of plunger elements cut from a single wire. The devices, systems and methods are especially useful for facilitating implantation of relatively fragile, for example brittle, ocular implants in an intact condition.

### BACKGROUND

The present invention generally relates to systems and methods for treating eyes and more specifically relates to systems and methods for delivering drug delivery implants into eyes, for example, mammalian eyes.

Solid pharmaceutically active implants that provide controlled release, for example, sustained release, of an active ingredient are able to provide a relatively uniform concentration of active ingredients in the body. Implants are particularly useful for providing a high local concentration at a particular target site for extended periods of time. Additionally, sustained release forms may reduce the number of doses of the drug required to be effective in treatment of a condition, and often reduce the occurrence of side effects and/or inconsistency in drug concentration found with traditional drug therapies.

Various sites exist in the eye for implantation of a drug delivery device or implant, such as the vitreous of the eye, anterior or posterior chambers of the eye, or other areas of the eye including intraretinal, subretinal, intrachoroidal, suprachoroidal, intrascleral, episcleral, subconjunctival, intracorneal or epicorneal spaces. Wherever the desired location of implantation, typical methods of implantation all require relatively invasive surgical procedures, pose a risk of excessive trauma to the eye, and require excessive handling of the implant. For example, in a typical method for placement in the vitreous, an incision is made through the sclera, and the implant is inserted into and deposited at the desired location in the vitreous, using forceps or other like manual grasping device. Once deposited, the forceps (or grasping device) is removed, and the incision is sutured closed.

There are many drawbacks of such techniques for intraocular implant delivery. Extensive handling of the implant is necessitated in these techniques, creating a risk that the implant will be damaged in the process. Many such implants, particularly bioerodible implants, are polymer based and are relatively fragile. If portions of such implants are damaged and broken-off, the effective therapeutic dose delivered by the implant once placed will be significantly altered. In addition, it becomes inherently difficult using these methods to achieve reproducible placement from patient to patient.

There thus remains a need for a more convenient, less invasive, and less traumatic means for delivering implants into the eye, for example, into the vitreous of the eye. There also remains a need for a more controlled means of delivering implants into the eye.

The following patents and additional publications include disclosure which is relevant to and/or helpful in understanding the present invention.

Weber et al., U.S. patent application Ser. No. 10/246,884, filed on Sep. 18, 2002, having Pub. No. U.S. 2004/0054374 A1, describes apparatus and methods for delivering ocular implants into an eye of a patient.

Wong, U.S. Pat. No. 4,997,652 discloses biodegradable ocular implants, including encapsulated agents, and describes implanting microcapsules comprising hydrocortisone succinate into the posterior segment of the eye.

Wong, U.S. Pat. No. 5,164,188 discloses encapsulated agents for introduction into the suprachoroid of the eye, and describes placing microcapsules and plaques comprising hydrocortisone into the pars plana.

Wong et al., U.S. Pat. Nos. 5,443,505 and 5,766,242 disclose implants comprising active agents for introduction into a suprachoroidal space or an avascular region of the eye, and describes placing microcapsules and plaques comprising hydrocortisone into the pars plana.

Wong et al., U.S. Pat. No. 5,869,079 discloses combinations of hydrophilic and hydrophobic entities in a biodegradable sustained release implant, and describes a polylactic acid polyglycolic acid (PLGA) copolymer implant comprising dexamethasone.

Wong, U.S. Pat. No. 5,824,072 discloses implants for introduction into a suprachoroidal space or an avascular region of the eye, and describes a methylcellulose (i.e. non-biodegradable) implant comprising dexamethasone.

Zhou et al. disclose a multiple-drug implant comprising 5-fluorouridine, triamcinolone, and human recombinant tissue plasminogen activator for intraocular management of proliferative vitreoretinopathy. Zhou, T., et al. “Development of a multiple-drug delivery implant for intraocular management of proliferative vitreoretinopathy”, Journal of Controlled Release 55: pp. 281-295.

Heller, “Biodegradable Polymers in Controlled Drug Delivery”, in: CRC Critical Reviews in Therapeutic Drug Carrier Systems, Vol. 1, (CRC Press, Boca Raton, Fla., 1987), pp 39-90, describes encapsulation for controlled drug delivery. Heller, in: Hydrogels in Medicine and Pharmacy, N. A. Peppes ed., Vol. III, (CRC Press, Boca Raton, Fla., 1987), pp 137-149, describes bioerodible polymers.

Anderson et al., Contraception 13:375, (1976), and Miller et al., J. Biomed. Materials Res. 11:711, (1977) describe various properties of poly(dL-lactic acid).

Brine, U.S. Pat. No. 5,075,115 discloses controlled release formulations with lactic acid polymers and co-polymers.

Di Colo, Biomaterials 13:850-856 (1992) describes controlled drug release from hydrophobic polymers.

Olejnik, et al. U.S. Pat. No. 6,074,661 discloses an implantable device for treatment of an eye, wherein the device incorporates a retinoid for improving the biocompatability of the device in eye tissue.

Wong, U.S. Pat. No. 6,699,493 discloses a method for reducing or preventing transplant rejection in the eye and intraocular implants for use therefor.

Other documents that are also relevant or otherwise helpful in understanding the present invention are U.S. patent application Ser. No. 09/693,008, filed on Jul. 5, 2000; Ser. No. 10/327,018, filed on Dec. 20, 2002 and Ser. No. 10/340,237, filed on Jan. 9, 2003.

The entire disclosure of each of the documents cited hereinabove is incorporated herein in its entirety by this reference.

### SUMMARY

The present invention provides new devices, systems and methods for placing a drug delivery implant into an eye, for example, to achieve one or more desired therapeutic effects. The present apparatus and methods are useful for placing drug delivery implants, for example, substantially biodegradeable drug delivery implants, into an eye, such that the implant remains substantially intact during the placement procedure, for example, without causing any substantial breakage or other damage to the implant. For example, the devices, systems and methods of the invention are suitable for use in implanting drug delivery implants that are relatively non-flexible, rigid, fragile, and/or otherwise prone to damage or breakage. For example, the present methods and systems are highly effective in delivering an implant in an intact condition into an eye wherein the implant has a degree of brittleness that would typically contribute to breakage thereof in the event the same implant had been subjected to a conventional surgical implantation procedure.

For example, in a preferred embodiment of the invention, the hollow sleeve is structured to accommodate the implant in a substantially frictionless manner, meaning that a degree of friction between the implant and the inner wall of the sleeve is in effect negligible. Advantageously, in some embodiments of the invention, the hollow sleeve is made of a fluoroplastic material, or other like material In these embodiments, the device is structured such that frictional forces generated upon movement between the relatively delicate, brittle implant and the sleeve will not be sufficient to cause any substantial damage to the implant as the implant is passed through the hollow sleeve and into the eye.

In a broad aspect of the invention, methods for treating an eye are provided. Such methods generally comprise providing a device comprising a hollow sleeve having an open proximal end, a distal end, a substantially uniform inner diameter and a substantially uniform outer diameter extending between the proximal end and the open distal end. The method further comprises placing an implant, for example, a bioerodible or biodegradable therapeutic implant, in the hollow sleeve, preferably at or near the distal end thereof. The method further comprises advancing the distal end of the hollow sleeve through the surface of the eye and passing the implant out of the distal end of the hollow sleeve. The hollow sleeve is then withdrawn from the eye, leaving the implant in place within the eye.

Advantageously, the method may be performed without provision of an initial incision. For example, the step of advancing the distal end through the surface of the eye may comprise placing a distal edge of the hollow sleeve adjacent the surface of the eye, puncturing the eye with the distal edge, and passing the distal end into the eye at a desired location within the eye.

In some embodiments of the invention, withdrawal of the device from the eye leaves a puncture or perforation wound that is self-sealing in that no suturing or other like means for closing the wound is required.

Preferably, the method of the present invention is performed by direct manual manipulation of the hollow sleeve, particularly by direct manual manipulation of the proximal end of the hollow sleeve, without use or provision of a handpiece, housing, or other structure fixed to the hollow sleeve.

Preferably, the step of introducing the implant into the eye includes advancing a plunger element, for example a relatively thin, elongated filament or wire, into the open proximal end of the hollow sleeve and passing the plunger element through the hollow sleeve in a distal direction in order to pass or urge the implant out of the distal end of the hollow sleeve. For example, the plunger element may be held in a relatively fixed position while the hollow sleeve is gently drawn in a proximal direction out of the eye. Thus, the plunger element functions to hold the implant in place while the hollow sleeve is withdrawn. The plunger element is then gently withdrawn from the eye, thereby leaving the implant in place within the eye.

Typically, the present invention is directed toward, but not limited to, treatment of a mammalian eye, for example an animal or a human eye. In other aspects of the invention, ocular research systems and methods are provided that can be incorporated into research and testing of drug delivery implants in animal studies. It is also contemplated that the systems and methods of the present invention are particularly advantageous for clinical treatment of large animals, for example, large wild or domestic animals such as horses.

In some embodiments of the invention, the implant is placed in one of the anterior chamber or the posterior chamber of the eye. In other embodiments of the invention, the implant is placed in a structure of the eye selected from the group consisting of the retina, the vitreous, the cornea, and the scleral of the eye. In other embodiments of the invention the implant is passed into a structure of the eye consisting of the meningeal space, the optic nerve, and the intraoptic nerve.

The systems and methods of the invention are especially useful for implanting therapeutically active implants for example therapeutically active implants that are at least partially biodegradable or bioerodible. In some embodiments of the invention in which the implant is substantially entirely biodegradable or bioerodible, removal of the implant, after substantially complete active component release, is not required.

In one broad aspect of the invention, the devices, systems and methods of the present invention include implanting into an eye a biodegradable or bioerodible controlled release drug delivery implant, including a therapeutic component, sometimes referred to elsewhere herein as an “active component” or a “therapeutically active component”, and a matrix component, for example a polymeric matrix material.

It will be appreciated that the implant compositions will vary according to the ocular condition being treated, the preferred drug release profile, the particular active agent used, and the medical history of the patient.

For example, in some embodiments of the present invention, the implant advantageously comprises a therapeutic component admixed with one or more matrix materials, for example, one or more polymeric materials, for example one or more biodegradable polymeric materials. The implant may be structured to provide a controlled rate of release of the active agent therefrom upon erosion or degradation of an inactive, bioerodible or biodegradable material, for example polymeric material mixed with the therapeutic agent.

The devices, systems and methods of the present invention can be used to deliver any desired therapeutic agent, or combination of therapeutic agents, including an antibiotic agent, an antiviral agent, an antifungal agent, an anti-cancer agent, an antiglaucoma agent, an antiinflammatory agent, an analgesic, an immunomodulatory agent, a macro-molecule, or a mixture thereof.

Therapeutic, active agents that may be used in the systems and methods of the present invention include, but are not limited to ace-inhibitors, endogenous cytokines, agents that influence basement membrane, agents that influence the growth of endothelial cells, adrenergic agonists or blockers, cholinergic agonists or blockers, aldose reductase inhibitors, analgesics, anesthetics, antiallergics, anti-inflammatory agents, antihypertensives, pressors, antibacterials, antivirals, antifungals, antiprotozoals, anti-infectives, antitumor agents, antimetabolites, antiangiogenic agents, tyrosine kinase inhibitors, antibiotics such as aminoglycosides such as gentamycin, kanamycin, neomycin, and vancomycin; amphenicols such as chloramphenicol; cephalosporins, such as cefazolin HCl; penicillins such as ampicillin, penicillin, carbenicillin, oxycillin, methicillin; lincosamides such as lincomycin; polypeptide antibiotics such as polymixin and bacitracin; tetracyclines such as tetracycline; quinolones such as ciproflaxin, etc.; sulfonamides such as chloramine T; and sulfones such as sulfanilic acid as the hydrophilic entity, anti-viral drugs, e.g. acyclovir, gancyclovir, vidarabine, azidothymidine, dideoxyinosine, dideoxycytosine, dexamethasone, ciproflaxin, water soluble antibiotics, such as acyclovir, gancyclovir, vidarabine, azidothymidine, dideoxyinosine, dideoxycytosine; epinephrine; isoflurphate; adriamycin; bleomycin; mitomycin; ara-C; actinomycin D; scopolamine; and the like, analgesics, such as codeine, morphine, keterolac, naproxen, etc., an anesthetic, e.g. lidocaine; .beta.-adrenergic blocker or .beta.-adrenergic agonist, e.g. ephidrine, epinephrine, etc.; aldose reductase inhibitor, e.g. epalrestat, ponalrestat, sorbinil, tolrestat; antiallergic, e.g. cromolyn, beclomethasone, dexamethasone, and flunisolide; colchicine, anihelminthic agents, e.g. ivermectin and suramin sodium; antiamebic agents, e.g. chloroquine and chlortetracycline; and antifungal agents, e.g. amphotericin, etc., anti-angiogenesis compounds such as anecortave acetate, retinoids such as Tazarotene, anti-glaucoma agents, such as brimonidine (Alphagan and Alphagan P), acetozolamide, bimatoprost (Lumigan), Timolol, mebefunolol; memantine; alpha-2 adrenergic receptor agonists; 2ME2; anti-neoplastics, such as vinblastine, vincristine, interferons; alpha., beta. and .gamma., antimetabolites, such as folic acid analogs, purine analogs, and pyrimidine analogs; immunosuppressants such as azathiprine, cyclosporine and mizoribine; miotic agents, such as carbachol, mydriatic agents such as atropine, etc., protease inhibitors such as aprotinin, camostat, gabexate, vasodilators such as bradykinin, etc., and various growth factors, such epidermal growth factor, basic fibroblast growth factor, nerve growth factors, and the like.

The devices, systems and methods of the present invention are generally useful for implanting drug delivery implants in the form of fibers, rods, filaments, pellets, granules, and the like, or may be of any size or shape compatible with the selected site of implantation, as long as the implants can be received within and passed through the hollow sleeve and have the desired release kinetics and deliver an amount of active agent that is therapeutic for the intended medical condition of the eye.

The upper limit for the implant size will be determined by factors such as the desired release kinetics, toleration for the implant at the site of implantation, size limitations on insertion, and ease of handling. For example, the vitreous chamber is able to accommodate relatively large rod-shaped implants, generally having diameters of about 0.05 mm to 3 mm and a length of about 0.5 to about 10 mm. In one variation, the rods have diameters of about 0.1 mm to about 1 mm. In another variation, the implants comprise rods having diameters of about 0.3 mm to about 0.75 mm. In yet a further variation, other implants having variable geometries but approximately similar volumes may also be used.

The devices, systems and methods of the invention may comprise introducing a plurality of biodegradable drug delivery implants into the same or different chambers or the same or different structures of the eye.

The implant may include a single therapeutic agent or a plurality of different therapeutic agents depending upon the nature of the condition or conditions of the eye being treated. The site of implantation can vary depending upon the ocular condition being treated and the desired course of treatment.

For example, the present devices, systems and methods may be designed to be effective in treating an inflammation mediated condition, for example, uveitis. In this case, an implant is provided including a therapeutic component comprising an anti-inflammatory agent, and a method of the invention may include placing the implant proximal to the uveal structures.

In another example, the present devices, systems and methods may be structured for treatment of glaucoma. An implant may be provided which is structured to provide sustained release of one or more neuroprotective agents that protect cells from excitotoxic damage. The implant may be alternatively or additionally structured to be effective in delivering one or more beta-blockers, for example Timolol Maleate, to the eye on a substantially consistent basis. Other agents include -methyl-D-aspartate (NMDA) antagonists, cytokines, and neurotrophic factors. The methods and devices are designed and structured to deliver one of more such implants deep into the vitreous.

In yet another example, the present devices, systems and methods may be structured for treatment of diabetic retinopathy. The therapeutic component may comprise one or more anti-angiogenic agents and/or one or more neurotropic agents, and the implant may be placed within the vitreous using the devices or methods of the invention.

In yet another example, the present devices, systems and methods may be structured for treating age-related macular degeneration. For example, the devices, systems and methods are useful for delivering implants including one or more neurotrophic factors preferably to the vitreous, and/or one or more anti-angiogenic factors intraocularly or periocularly, preferably periocularly, most preferably to the sub-Tenon's region.

Each and every feature described herein, and each and every combination of two or more of such features, is included within the scope of the present invention provided that the features included in such a combination are not mutually inconsistent.

Additional aspects and advantages of the present invention are set forth in the following description and claims, particularly when considered in conjunction with the accompanying drawings in which like parts bear like reference numerals.

### DRAWINGS

FIG. 1 shows a cross sectional view of a device useful for performing a method of the present invention.

FIG. 2 shows an embodiment of the present invention comprising a plurality of hollow sleeves and a plurality of plunger elements, useful for performing a method of the present invention.

FIG. 3 shows a cross-sectional view of an eye.

### DESCRIPTION

The invention provides devices and methods for delivering biodegradable ocular implants into a region of an eye, for example a vitreous, an anterior chamber, a posterior chamber, or other location within an eye. The present devices and methods are suitable for treatment of ocular conditions, such as an anterior ocular condition, a posterior ocular condition, or an ocular condition which can be characterized as both an anterior ocular condition and a posterior ocular condition.

As used herein, and as generally understood by those of skill in the art, an ocular condition can include a disease, aliment or condition which affects or involves the eye or one of the parts or regions of the eye. Broadly speaking, the eye includes the eyeball and the tissues and fluids which constitute the eyeball, the periocular muscles (such as the oblique and rectus muscles) and the portion of the optic nerve which is within or adjacent to the eyeball.

An anterior ocular condition is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves, the conjunctiva, the cornea, the conjunctiva, the anterior chamber, the iris, the posterior chamber (behind the retina but in front of the posterior wall of the lens capsule), the lens or the lens capsule and blood vessels and nerve which vascularize or innervate an anterior ocular region or site. An anterior ocular condition can include a disease, ailment or condition, such as for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye.

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site. Thus, a posterior ocular condition can include a disease, ailment or condition, such as for example, macular degeneration (such as non-exudative age related macular degeneration and exudative age related macular degeneration); choroidal neovascularization; acute macular neuroretinopathy; macular edema (such as cystoid macular edema and diabetic macular edema); Behcet's disease, retinal disorders, diabetic retinopathy (including proliferative diabetic retinopathy); retinal arterial occlusive disease; central retinal vein occlusion; uveitic retinal disease; retinal detachment; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy; non-retinopathy diabetic retinal dysfunction, retinitis pigmentosa and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

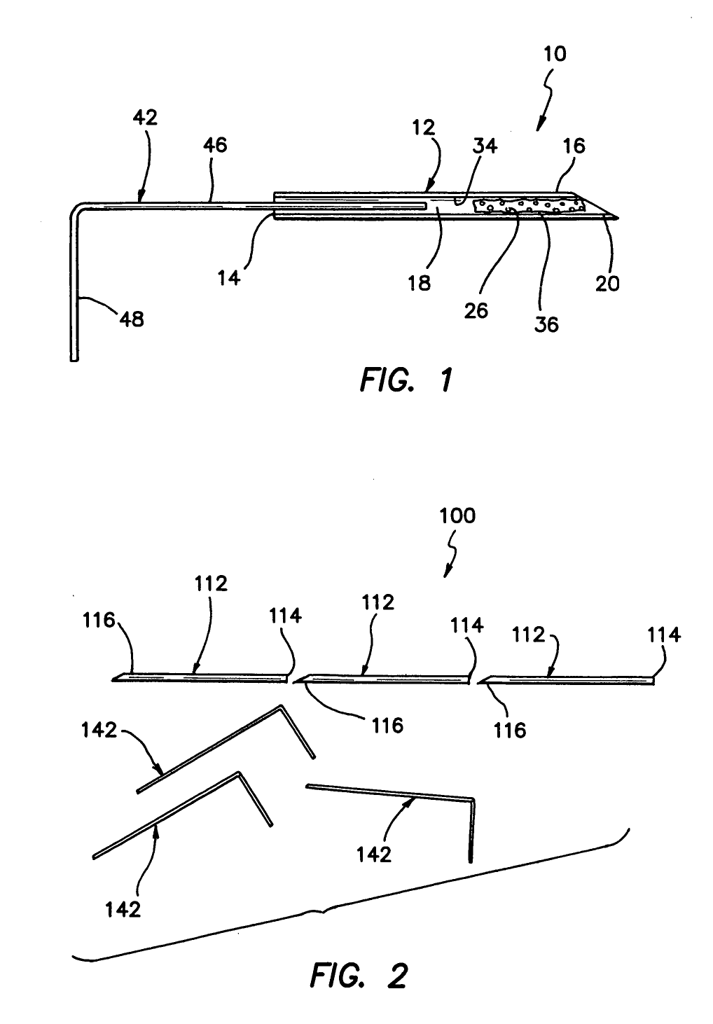

Referring now to FIG. 1, a device for treating eyes, for example mammalian eyes, in accordance with the present invention, is shown generally at 10.

The device 10 generally comprises a hollow sleeve 12 having an open proximal end 14, a distal end 16, a substantially uniform inner diameter defining a lumen 18, and a substantially uniform outer diameter extending between the proximal end 14 and the distal end 16, the hollow sleeve 12 terminating at the proximal end 14. The hollow sleeve 12 may further comprise a beveled distal tip 20.

When the device 10 is used to perform a method of the invention, an implant or element, for example a biodegradable implant 26 is placed in the lumen 18, for example, proximally the beveled distal tip 20 as shown.

The implant 26 generally includes a therapeutic component 28 and preferably a matrix component 32, and is sized to be received, for example, slidably received, within the sleeve lumen 18. Even more preferably, the implant 26 is structured, for example sized, to be received within the lumen 18 with negligible frictional contact between an inner wall 34 of the hollow sleeve 12 and an outer surface 36 of the implant 26.

As used herein, “implants” refers to ocular implants or drug delivery devices which can be implanted into any number of locations in an eye and which are designed such that a controlled amount of desired drug or therapeutic agent can be released over time. Such implants are biocompatible, and in many cases are formed of a bioerodible substance, such as a bioerodible polymer. Such implants may have a sufficiently small cross-sectional area that they can be delivered by the devices, methods and systems of the present invention in such a manner that results in self-sealing of the eye at the puncture site associated with the delivery procedure. In other words, methods of delivering implants into the eye in accordance with some embodiments of the invention, do not require a suture or other like closing means at the puncture site. Such methods in accordance with the invention tend to minimize initial leakage at the puncture site to a degree that a surgeon, or another person equally skilled in the art, in his or her good clinical judgment, would not be compelled to suture or otherwise provide other like closure means to the puncture site.

In particular, such implants, also sometimes more specifically referred to herein as “microimplants” have dimensions such that they are deliverable through a hollow sleeve having a size equivalent to a 21 gauge or 22 gauge or smaller needle or cannula. Thin walled versions of 22 gauge needles or cannulas can have inner diameters of less than about 0.028 inches, and thus cylindrical microimplants deliverable through such similarly sized hollow sleeves will have outer diameters of less than about 0.028 inches. Thin walled versions of 22 gauge needles or cannulas can have inner diameters of up to about 0.023 inches, and thus cylindrical microimplants with diameters of less than about 0.023 inches can be delivered through similarly sized hollow sleeves.

The hollow sleeve 12 may comprise any suitable material, for example, stainless steel. More preferably, the hollow sleeve comprises a polymeric material, for example, a fluoroplastic material. In some embodiments of the invention, the hollow sleeve comprises a material selected from the group consisting of polytetrafluoroethylene, polyperfluoroalkoxyl, ethylene propylene, polyfluorinated ethylene propylene, and combinations thereof.

The device 10 may further comprise a plunger element 42 structured to be slidably received within the hollow sleeve 12, and structured to urge the implant 26 out from the distal end 16 of the hollow sleeve 12. For example, the plunger element 42 may comprise a thin filament, or other elongated element, for example, a bendable wire for example, a stainless steel wire, having an outer diameter less than the inner diameter of the hollow sleeve 12. In the particular embodiment of the invention shown in FIG. 1, the plunger element 42 includes a distal portion 46 with a length about equal to a length of the hollow sleeve 12, for example, no greater than the length of the hollow sleeve 12, and a proximal portion 48 disposed at an angle, for example an angle of about 90 degrees, with respect to the distal portion 46. As shown, the plunger element 42 may have a substantially uniform diameter extending along the proximal portion 48 and the distal portion 46.

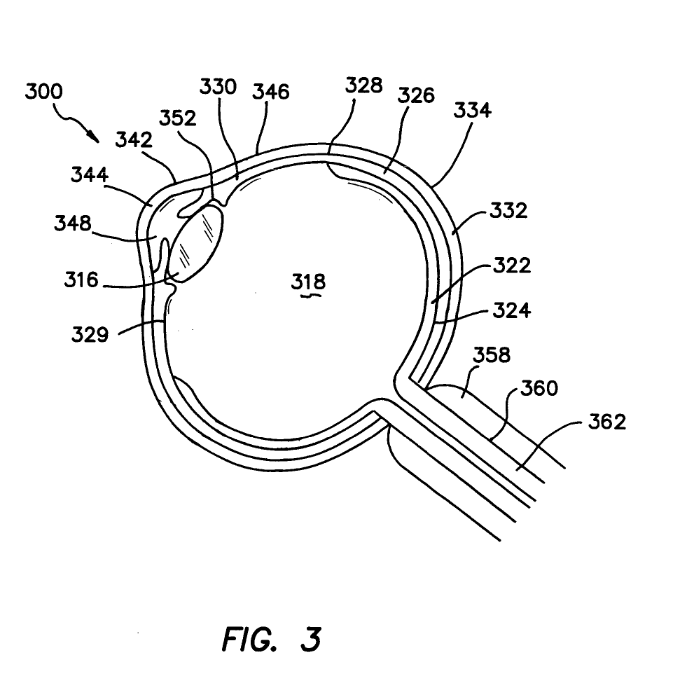

In use, the distal end 16 of the hollow sleeve 12, having the implant 26 disposed therein, is passed through a surface of an eye, for example, such as the eye 300 shown in FIG. 3. The implant 26 is then introduced into the eye at a desired location within the eye by passing the implant 26 out of the distal end 16 of the hollow sleeve 12. The hollow sleeve 12 is withdrawn from the eye, and the implant is left in place therein.

More specifically, the distal portion 46 of the plunger element 42 is passed into the open proximal end 14 of the sleeve 12 until the far distal edge of the plunger element 46 abuts the implant 26. This may be accomplished by puncturing the eye through the sclera with the sharp beveled tip 20 of the hollow sleeve 12. The sleeve 12 and implant 26 are then advanced into the vitreous to an appropriate position within the eye. Determination of the depth of the distal end 16 within eye can be facilitated by suitable indicia or markings placed on the sleeve at specific spaced apart intervals. The sleeve 12 may then be withdrawn from the eye while the plunger element 42 is used to hold the implant 26 in a substantially stationary position within the eye. The plunger element 42 may then be withdrawn from the eye, thereby leaving the implant 26 in place. The puncture wound may be self-sealing as described elsewhere herein, or may be sutured or otherwise closed.

Advantageously, it can be appreciated that the device 10 is structured to enable use thereof by means of direct manual manipulation of the hollow sleeve 12 and plunger element 42. For example, no additional structure, such as a housing, handpiece, holder or like structure is necessary for effective use of the present invention.

Turning now to FIG. 2, another embodiment of the invention is shown. More particularly, the present invention may further provide an ocular research system 100 comprising a plurality of hollow sleeves 112, sized to be inserted into an eye, for example a vitreous of an eye. Each sleeve 112 may be substantially identical to each other sleeve 112. Each sleeve 112 includes an open proximal end 114, a distal end 116, and a substantially uniform inner diameter and a substantially uniform outer diameter extending between the proximal end 114 and the distal end 116. The plurality of hollow sleeves 112 may be produced by cutting a single tube of material, at preferably equally spaced apart intervals.

As shown, the system 100 further comprises a plurality of plunger elements 142, wherein each plunger element 142 is structured to be slidably received within one of the hollow sleeves 112.

Except as expressly described herein, each sleeve 112 and each plunger element 142 in system 100 is substantially identical to sleeve 12 and plunger element 42, respectively, of device 10 shown in FIG. 1. Features of sleeve 112 and plunger element 142 which correspond to features of sleeve 12 and plunger element 42 are designated by the corresponding reference numerals increased by 100.

In some embodiments of the present invention, a kit is provided. More particuallry, for example, an equal number of hollow sleeves 112 and plunger elements 142 may be packaged in a sterile material and provided, as a kit, to research and development facilities for performing ocular research trials and tests.

The system 100 may further comprise a plurality of ocular implants (not shown in FIG. 2), for example, a plurality of substantially biodegradable ocular implants each ocular implant including a therapeutic component and a matrix component and being sized to be slidably received within one of the hollow sleeves 112.

The system 100 is especially useful for performing ocular research in animal studies, particularly in animal studies for testing biodegradable ocular implants. Polymer based implants that are relatively fragile, brittle non-flexible or rigid are difficult to implant, especially in an intact condition, into an eye, particularly into a posterior section of an eye. The present systems are designed to maintain the implants in an intact condition during implantation procedure, reduce trauma to the eye, promote more consistent test results, and generally facilitate these types of animal research studies.

### EXAMPLE

A plurality of hollow sleeves 112 were made from a single, standard wall, 28 grade, 0.015 inch inner diameter Teflon® tube. The tube was marked and cut into increments of about 4.0 cm each increment. Using a suitable surgical blade, each increment was made into a sharpened hollow sleeve 112 by cutting one edge of each increment at a 45 degree angle, to form a sharp edge, or distal tip 120, having about a 2.5 mm to about a 3.0 mm bevel. Each of the sharpened edges was marked for convenience.

A plurality of plunger elements 142 were made from a single piece of stainless steel wire that was marked and cut at increments of about 5.5 cm. Using pliers, individual stainless steel plunger elements 142 were formed by bending each wire increment at a 90 degree angle with a proximal portion measuring about 1.5 cm, and a distal portion measuring about 4.0 cm.

These devices, each device comprising one of the hollow sleeves 112 and one of the plunger elements 142, were used in rabbit model animal studies for testing biodegradable intraocular implants having cross-sectional diameters of less than 0.015 inches, and comprising the anti-inflammatory steroid dexamethasone, and a co-polymer matrix of lactic acid and glycolic acid. The devices were used to implant the intraocular implants into the vitreous, of one eye of each rabbit in the study.

In other embodiments of the invention, the devices, systems and methods are useful for effective clinical treatment of an eye of a domestic animal, for example an equine eye. The present systems and methods can provide effective means of quickly delivering a drug into a posterior chamber of an eye of such an animal with minimal trauma and little or no significant injury being inflicted on the eye of the animal. It is contemplated that the present systems and methods are particularly advantageous for use in treatment of a posterior chamber condition, for example, but not limited to, treatment of a detached retina in a horse or other large animal.

Implants useful with the present invention include therapeutically active implants comprising a therapeutic component and a polymeric matrix component.

Suitable polymeric materials or compositions for use in the implants include those materials which are compatible, that is biocompatible, with the eye so as to cause no substantial interference with the functioning or physiology of the eye.

The matrix component may comprise materials which are at least partially, for example, are substantially completely, biodegradable or bioerodible, when exposed to the ocular environment. Once implanted in the eye, the matrix material degrades within the eye, releasing the therapeutic component into the eye, and providing substantially consistent, for example, substantially constant therapeutic benefit thereto.

In other embodiments of the invention, the matrix component 26 is made of materials that are not biodegradeable, or are not substantially biodegradable, when exposed to the ocular environment.

In the present context, a biodegradable or bioerodible material is one which degrades into physiologically acceptable degradation products under physiological conditions in the eye, or erodes into physiologically acceptable materials under physiological conditions in the eye.

The implants may be structured such that the biodegradable polymer matrix may comprise at least about 10 percent, at least about 20 percent, at least about 30 percent, at least about 40 percent, at least about 50 percent, at least about 60 percent, at least about 70 percent, at least about 80 percent, at least about 90 percent of the implant.

Biodegradable polymers which can be used in the implants include, but are not limited to, polymers made of monomers such as organic esters or ethers, which when degraded result in physiologically acceptable degradation products. Anhydrides, amides, orthoesters, or the like, by themselves or in combination with other monomers, may also be used. The polymers are generally condensation polymers. The polymers can be crosslinked or non-crosslinked. If crosslinked, they are usually not more than lightly crosslinked, and are less than 5% crosslinked, usually less than 1% crosslinked.

For the most part, besides carbon and hydrogen, the polymers will include oxygen and nitrogen, particularly oxygen. The oxygen may be present as oxy, e.g., hydroxy or ether, carbonyl, e.g., non-oxo-carbonyl, such as carboxylic acid ester, and the like. The nitrogen can be present as amide, cyano, and amino. An exemplary list of biodegradable polymers that can be used are described in Heller, “Biodegradable Polymers in Controlled Drug Delivery”, in: CRC Critical Reviews in Therapeutic Drug Carrier Systems, Vol. 1. (CRC Press, Boca Raton, Fla., 1987).

Of particular interest are polymers of hydroxyaliphatic carboxylic acids, either homo- or copolymers, and polysaccharides. Included among the polyesters of interest are homo- or copolymers of D-lactic acid, L-lactic acid, racemic lactic acid, glycolic acid, caprolactone, and combinations thereof. Copolymers of glycolic and lactic acid are of particular interest, where the rate of biodegradation is controlled by the ratio of glycolic to lactic acid. The percent of each monomer in poly(lactic-co-glycolic)acid (PLGA) copolymer may be 0-100%, about 15-85%, about 25-75%, or about 35-65%. In certain variations, 25/75 PLGA and/or 50/50 PLGA copolymers are used. In other variations, PLGA copolymers are used in conjunction with polylactide polymers.

Biodegradable polymer matrices that include mixtures of hydrophilic and hydrophobic ended PLGA may also be employed, and are useful in modulating polymer matrix degradation rates. Hydrophobic ended (also referred to as capped or end-capped) PLGA has an ester linkage hydrophobic in nature at the polymer terminus. Typical hydrophobic end groups include, but are not limited to alkyl esters and aromatic esters. Hydrophilic ended (also referred to as uncapped) PLGA has an end group hydrophilic in nature at the polymer terminus. PLGA with a hydrophilic end groups at the polymer terminus degrades faster than hydrophobic ended is PLGA because it takes up water and undergoes hydrolysis at a faster rate (Tracy et al., Biomaterials 20:1057-1062 (1999)). Examples of suitable hydrophilic end groups that may be incorporated to enhance hydrolysis include, but are not limited to, carboxyl, hydroxyl, and polyethylene glycol. The specific end group will typically result from the initiator employed in the polymerization process. For example, if the initiator is water or carboxylic acid, the resulting end groups will be carboxyl and hydroxyl. Similarly, if the initiator is a monofunctional alcohol, the resulting end groups will be ester or hydroxyl.

The composition of the implants may be monolithic, that is, having the therapeutic component substantially uniformly distributed throughout the matrix component, for example, throughout the polymeric material present in the implant, or the implants may have encapsulated reservoirs for example, particles and/or other relatively concentrated forms, of therapeutic component interspersed throughout the implant, for example, throughout the polymeric material in the implant.

Among the useful polysaccharides are, without limitation, calcium alginate, and functionalized celluloses, particularly carboxymethylecellulose esters characterized by being water insoluble, a molecular weight of about kD to 500 kD, etc.

Other polymers of interest include, without limitation, polyvinyl alcohol, polyesters, polyethers and combinations thereof which are biocompatible and may or may not be biodegradable and/or bioerodible.

Some preferred characteristics of the polymers or polymeric materials for use in the present invention may include biocompatibility, compatibility with the therapeutic component, ease of use of the polymer in making the drug delivery systems of the present invention, a halflife in the physiological environment of at least about 6 hours, preferably greater than about one day, not significantly increasing the viscosity of the vitreous, and water insolubility.

The biodegradable polymeric materials are desirably subject to enzymatic or hydrolytic instability. Water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, where the polymers may be employed as varying layers or mixed.

Alternatively or additionally, various non-biodegradable polymeric compositions may be employed in the implants. The non-biodegradable polymeric composition employed may allow for release of the drug by, for example, solution/diffusion or leaching mechanisms. The non-biodegradable polymeric compositions employed may be varied according to the compatibility of the polymer with the drug or other active agent to be employed, ease of manufacture, the desired rate of release of the drug, desired density or porosity, and the like. Various non-biodegradable polymers which may be employed are described in U.S. Pat. Nos. 4,303,637; 4,304,765; 4,190,642; 4,186,184; 4,057,619; 4,052,505; 4,281,654; 4,959,217; 4,014,335; 4,668,506; 4,144,317. The non-biodegradable polymers may be homopolymers, copolymers, straight, branched-chain, or cross-linked derivatives.

Exemplary biocompatible, non-biodegradable polymers of particular interest include polycarbamates or polyureas, particularly polyurethanes, polymers which may be cross-linked to produce non-biodegradable polymers such as cross-linked poly(vinyl acetate) and the like. Also of particular interest are ethylene-vinyl ester copolymers having an ester content of 4 to 80% such as ethylene-vinyl acetate (EVA) copolymer, ethylene-vinyl hexanoate copolymer, ethylene-vinyl propionate copolymer, ethylene-vinyl butyrate copolymer, ethylene-vinyl pentantoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer, and ethylene-vinyl benzoate copolymer, Ethylene-vinyl ester copolymers including ethylene-vinyl acetate copolymers for the manufacture of diffusional ocular drug delivery devices where the drug dissolves in and passes through the polymer by diffusion are described in U.S. Pat. Nos. 4,052,505 and 4,144,317.

Additional exemplary naturally occurring or synthetic non-biodegradable polymeric materials include poly(methylmethacrylate), poly(butylmethacrylate), plasticized poly(vinylchloride), plasticized poly(amides), plasticized nylon, plasticized soft nylon, plasticized poly(ethylene terephthalate), natural rubber, silicone, poly(isoprene), poly(isobutylene), poly(butadiene), poly(ethylene), poly(tetrafluoroethylene), poly(-vinylidene chloride), poly(acrylonitrile), cross-linked poly(vinylpyrrolidone), poly(trifluorochloroethylene), chlorinated poly(ethylene), poly(4,4′-isopropylidene diphenylene carbonate), vinylidene chloride-acrylonitrile copolymer, vinyl chloridediethyl fumarate copolymer, silicone, silicone rubbers (especially the medical grade), poly(dimethylsiloxanes), ethylene-propylene rubber, silicone-carbonate copolymers, vinylidene chloride-vinyl chloride copolymer, vinyl chloride-acrylonitrile copolymer, vinylidene chloride-acrylonitrile copolymer, poly(olefins), poly(vinyl-olefins), poly(styrene), poly(halo-olefins), poly(vinyls), poly(acrylate), poly(methacrylate), poly(oxides), poly(esters), poly(amides), and poly(carbonates).

Biodegradable or non-biodegradable hydrogels may also be employed in the implants of the subject invention. Hydrogels are typically a copolymer material, characterized by the ability to imbibe a liquid. Exemplary non-biodegradable hydrogels which may be employed and methods of making these hydrogels are described in U.S. Pat. Nos. 4,959,217 and 4,668,506, herein incorporated by reference.

In addition to the controlled porosity of the implant, in some embodiments of the invention which employ a non-biodegradable polymer, the rate of release of the drug will be solution/diffusion controlled. The rate of diffusion of drug through the non-biodegradable polymer may be affected by drug solubility, polymer hydrophilicity, extent of polymer cross-linking, expansion of the polymer upon water absorption so as to make the polymer more permeable to the drug, and the like.

The therapeutic component useful in the present invention may include any suitable pharmacologically active agent or therapeutic agent which is beneficial when placed within the eye, for example, an agent of which extended, delayed, or otherwise controlled release thereof in the eye, is desirable. Pharmacologic or therapeutic agents which may find use in the present systems, include, without limitation, those disclosed in U.S. Pat. No. 4,474,451, columns 4-6 and U.S. Pat. No. 4,327,725, columns 7-8, which disclosures are incorporated herein by reference.

Pharmacological or therapeutic agents of interest include hydrocortisone (5-20 mcg/l as plasma level), gentamycin (6-10 mcg/ml in serum), 5-fluorouracil (about 0.30 mg/kg body weight in serum), sorbinil, IL-2, TNF, Phakan-a (a component of glutathione), thioloa-thiopronin, Bendazac, acetylsalicylic acid, trifluorothymidine, interferon (alpha., beta. and gamma.), immune modulators, e.g. lymphokines, monokines, and growth factors, etc.

Pharmacological or therapeutic agents of particular interest include, without limitation, anti-glaucoma drugs, such as the beta-blockers, such as timolol maleate, betaxolol and metipranolol; mitotics, such as pilocarpine, acetylcholine chloride, isofluorophate, demacarium bromide, echothiophate iodide, phospholine iodide, carbachol, and physostigimine; epinephrine and salts, such as dipivefrin hydrochloride; and dichlorphenamide, acetazolamide and methazolamide; anti-cataract and anti-diabetic retinopathy drugs, such as aldose reductase inhibitors, such as tolrestat, lisinopril, enalapril, and statil; thiol cross-linking drugs other than those considered previously; anti-cancer drugs, such as retinoic acid, methotrexate, adriamycin, bleomycin, triamcinoline, mitomycin, cis-platinum, vincristine, vinblastine, actinomycin-D, ara-c, bisantrene, CCNU, activated cytoxan, DTIC, HMM, melphalan, mithramycin, procarbazine, VM26, VP16, and tamoxifen; immune modulators, other than those indicated previously; anti-clotting agents, such as tissue plasminogen activator, urokinase, and streptokinase; anti-tissue damage agents, such as superoxide dismutase; proteins and nucleic acids, such as mono- and polyclonal antibodies, enyzmes, protein hormones and genes, gene fragments and plasmids; steriods, particularly anti-inflammatory or anti-fibrous drugs, such as cortisone, hydrocortisone, prednisolone, prednisome, dexamethasone, peogesterone-like compounds, medrysone (HMS) and fluorometholone; non-steroidal anti-inflammatory drugs, such as ketrolac tromethamine, dichlofenac sodium and suprofen; antibiotics, such as loridine (cephaloridine), chloramphenicol, clindamycin, amikacin, tobramycin, methicillin, lincomycin, oxycillin, penicillin, amphotericin B, polymyxin B, cephalosporin family, ampicillin, bacitracin, carbenicillin, cepholothin, colistin, erythromycin, streptomycin, neomycin, sulfacetamide, vancomycin, silver nitrate, sulfisoxazole diolamine, and tetracycline; other antipathogens, including anti-viral agents, such as idoxuridine, trifluorouridine, vidarabine (adenine arabinoside), acyclovir (acycloguanosine), pyrimethamine, trisulfapyrimidine-2, clindamycin, nystatin, flucytosine, natamycin, miconazole and piperazie derivatives, e.g. diethylcarbamazine; cycloplegic and mydriatic agents, such as atropine, cyclogel, scopolamine, homatropine and mydriacyl; and the like and mixtures thereof.

Other agents useful in the systems of the present invention include, without limitation, anticholinergics, anticoagulants, antifibrinolytic agents, antihistamines, antimalarials, antitoxins, chelating agents, hormones, immunosuppressives, thrombolytic agents, vitamins, salts, desensitizing agents, prostaglandins, amino acids, metabolites, antiallergenics, and the like and mixtures thereof.

In one embodiment of the invention, the active agent is methotrexate. In another embodiment, the active agent is a retinoic acid. In another embodiment, the active agent is an anti-inflammatory agent such as a nonsteroidal anti-inflammatory agent. Nonsteroidal anti-inflammatory agents that may be used include, but are not limited to, aspirin, diclofenac, flurbiprofen, ibuprofen, ketorolac, naproxen, and suprofen. In a further variation, the anti-inflammatory agent is a steroidal anti-inflammatory agent.

The steroidal anti-inflammatory agents that may be used in the implants include, but are not limited to, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and any of their derivatives.

In one aspect of the invention, cortisone, dexamethasone, fluocinolone, hydrocortisone, methylprednisolone, prednisolone, prednisone, and triamcinolone, and their derivatives, are preferred steroidal anti-inflammatory agents. In another aspect of the invention, the steroidal anti-inflammatory agent is dexamethasone. In another aspect of the invention, the biodegradable implant includes a combination of two or more steroidal anti-inflammatory agents.

The therapeutic component, for example, the active agent, can comprise from about 10% to about 90% by weight of the implant. In one variation, the active agent is from about 40% to about 80% by weight of the implant.

Other agents may be employed in the formulation for a variety of purposes. For example, buffering agents and preservatives may be employed. Preservatives which may be used include, but are not limited to, sodium bisulfite, sodium bisulfate, sodium thiosulfate, benzalkonium chloride, chlorobutanol, thimerosal, phenylmercuric acetate, phenylmercuric nitrate, methylparaben, polyvinyl alcohol and phenylethyl alcohol. Examples of buffering agents that may be employed include, but are not limited to, sodium carbonate, sodium borate, sodium phosphate, sodium acetate, sodium bicarbonate, and the like, as approved by the FDA for the desired route of administration. Electrolytes such as sodium chloride and potassium chloride may also be included in the formulation.

The implants can also include hydrophilic or hydrophobic compounds that accelerate or retard release of the active agent. Additionally, release modulators such as those described in U.S. Pat. No. 5,869,079 can be included in the implants. The amount of release modulator employed will be dependent on the desired release profile, the activity of the modulator, and on the release profile of the glucocorticoid in the absence of modulator. Where the buffering agent or release enhancer or modulator is hydrophilic, it may also act as a release accelerator. Hydrophilic additives act to increase the release rates through faster dissolution of the material surrounding the drug particles, which increases the surface area of the drug exposed, thereby increasing the rate of drug diffusion. Similarly, a hydrophobic buffering agent or enhancer or modulator can dissolve more slowly, slowing the exposure of drug particles, and thereby slowing the rate of drug diffusion.

In a particularly advantageous embodiment of the invention, devices, systems and methods suitable for treating inflammation-mediated conditions of the eye are provided. The term “inflammation-mediated condition of the eye” is meant to include any condition of the eye which may benefit from treatment with an anti-inflammatory agent, and is meant to include, but is not limited to, uveitis, macular edema, acute macular degeneration, retinal detachment, ocular tumors, fungal or viral infections, multifocal choroiditis, diabetic uveitis, proliferative vitreoretinopathy (PVR), sympathetic opthalmia, Vogt Koyanagi-Harada (VKH) syndrome, histoplasmosis, and uveal diffusion.

For example, the devices, systems and methods may comprise an implant, structured for being implanted into the vitreous of the eye wherein the therapeutic component comprises a steroidal anti-inflammatory agent, for example but not limited to, dexamethasone, and copolymer matrix of lactic acid and glycolic acid. The implant preferably delivers the agent to the vitreous in an amount sufficient to reach a concentration equivalent to at least about 0.05 μg/ml dexamethasone within about 48 hours and maintains a concentration equivalent to at least about 0.03 μg/ml dexamethasone for at least about three weeks. In another embodiment of the invention, the element 20 preferably delivers the agent to the vitreous in an amount sufficient to reach a concentration equivalent to at least about 0.2 μg/ml dexamethasone within about 6 hours and maintains a concentration equivalent to at least about 0.01 μg/ml dexamethasone for at least about three weeks.

“A concentration equivalent to dexamethasone”, as used herein, refers to the concentration of a steroidal anti-inflammatory agent necessary to have approximately the same efficacy in vivo as a particular dose of dexamethasone. For example, hydrocortisone is approximately twentyfivefold less potent than dexamethasone, and thus a 25 mg dose of hydrocortisone would be equivalent to a 1 mg dose of dexamethasone. One of ordinary skill in the art would be able to determine the concentration equivalent to dexamethasone for a particular steroidal anti-inflammatory agent from one of several standard tests known in the art. Relative potencies of selected corticosteroids may be found, for example, in Gilman, A. G., et al., eds. (1990). Goodman and Gilman's: The Pharmacological Basis of Therapeutics. 8th Edition, Pergamon Press: New York, p. 1447, which is incorporated herein by this specific reference.

In other embodiments, the implant delivers the agent to the vitreous in an amount sufficient to reach a concentration equivalent to at least about 0.3 μg/ml, or at least about 0.5 μg/ml, or at least about 0.75 μg/ml, or at least about 1.0 μg/ml, or at least about 2.0 μg/ml dexamethasone within about 4 hours, or within about 6 hours, or within about 8 hours, or within about 10 hours, or within about 24 hours.

A concentration equivalent to at least about 0.01 μg/ml, or at least about 0.02 μg/ml, or at least about 0.03 μg/ml, or at least about 0.05 μg/ml, or at least about 0.07 μg/ml dexamethasone may be maintained for an extended period of time (e.g., at least about three weeks or longer). The preferred concentration levels of therapeutic component or drug in the vitreous may vary according to the inflammatory mediated condition being treated. For example, for treating uveitis, a concentration equivalent of at least about 0.01 to 0.1 μg/ml dexamethasone is preferred.

In one embodiment, the concentration or therapeutic component is maintained for least about four weeks. In other embodiments, the concentration is maintained for at least about five weeks, or at least about six weeks, or at least about seven weeks, or at least about eight weeks, or at least about nine weeks, or at least about 10 weeks, or at least about 12 weeks or longer. The preferred duration of therapeutic component or drug release may be determined by the inflammatory mediated condition being treated. For treating uveitis, a drug release duration of at least about three weeks is preferable, more preferably at least about four weeks. In one embodiment, more than one implant or element 20 may be sequentially implanted into the vitreous in order to maintain therapeutic component or drug concentrations for even longer periods.

The formulation of the implants in accordance with the present invention may vary according to the desired therapeutic component release profile, the particular therapeutic component used, the condition being treated, and the medical history of the patient.

Parameters which determine the release kinetics from the implant include the size of the therapeutic component or drug particles entrapped in the element, the water solubility of the therapeutic component or drug, the ratio of therapeutic component or drug to polymer, the method of manufacture, the amount of surface area of the element exposed, the erosion rate of the polymer present in the element.

Preferably, the steroidal anti-inflammatory agent is selected from the group consisting of 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide and the like and mixtures thereof. In a preferred embodiment, the steroidal anti-inflammatory agent is selected from the group consisting of cortisone, dexamethasone, hydrocortisone, methylprednisolone, prednisolone, prednisone, triamcinolone and the like and mixtures thereof. In a more preferred embodiment, the steroidal anti-inflammatory agent is dexamethasone. In another embodiment, the bioerodible implant comprises more than one steroidal anti-inflammatory agent.

The amount or concentrations of therapeutic component employed in the element will vary depending on the effective dosage required and rate of release.

For embodiments of the invention employing steroidal anti-inflammatory agents, the polymers may comprise, for example, polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate.

Copolymers of glycolic and lactic acid are of particular interest, where the rate of biodegradation is controlled by the ratio of glycolic to lactic acid. The % of polylactic acid in the polylactic acid polyglycolic acid (PLGA) copolymer can be 0-100%, preferably about 15-85%, more preferably about 35-65%. In a particularly preferred embodiment, a 50/50 PLGA copolymer is used. The most rapidly degraded copolymer has roughly equal amounts of glycolic and lactic acid, where either homopolymer is more resistant to degradation. The ratio of glycolic acid to lactic acid will also affect the brittleness of in the element, where a more flexible element is desirable for larger geometries.

Other agents may be employed in the element 20 for a variety of purposes. In addition to the therapeutic component 23, effective amounts of buffering agents, preservatives and the like may be employed. Suitable water soluble preservatives include sodium bisulfite, sodium thiosulfate, ascorbate, benzalkonium chloride, chlorobutanol, thimerosal, phenylmercuric borate, parabens, benzyl alcohol, phenylethanol and the like and mixtures thereof. These agents may be present in amounts of from 0.001 to about 5% by weight and preferably 0.01 to about 2% by weight. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents advantageously present in amounts sufficient to maintain a pH of the system of between about 2 to about 9 and more preferably about 4 to about 8. As such, the buffering agent may be as much as about 5% by weight of the total implant or element 20.

Although the system 10 shown in FIG. 1 comprises hollow sleeve 12 and implant 26 both having corresponding substantially cylindrical cross-sectional forms, it is to be appreciated that the sleeve 12 and implant 26 may have other cross-sectional forms, for example triangular, rectangular, elliptical cross-sectional forms, are also included within the scope of the present invention.

The implant 26 may comprise an extruded filament having a size of between about 5 μm and about 2 mm, or between about 10 μm and about 1 mm.

Among the diseases/conditions which can be treated or addressed in accordance with the present invention include, without limitation, the following:

MACULOPATHIES/RETINAL DEGENERATION: Non-Exudative Age Related Macular Degeneration (ARMD), Exudative Age Related Macular Degeneration (ARMD), Choroidal Neovascularization, Diabetic Retinopathy, Acute Macular Neuroretinopathy, Central Serous Chorioretinopathy, Cystoid Macular Edema, Diabetic Macular Edema.

UVEITIS/RETINITIS/CHOROIDITIS: Acute Multifocal Placoid Pigment Epitheliopathy, Behcet's Disease, Birdshot Retinochoroidopathy, Infectious (Syphilis, Lyme, Tuberculosis, Toxoplasmosis), Intermediate Uveitis (Pars Planitis), Multifocal Choroiditis, Multiple Evanescent White Dot Syndrome (MEWDS), Ocular Sarcoidosis, Posterior Scleritis, Serpignous Choroiditis, Subretinal Fibrosis and Uveitis Syndrome, Vogt-Koyanagi-Harada Syndrome.

VASCULAR DISEASES/EXUDATIVE DISEASES: Retinal Arterial Occlusive Disease, Central Retinal Vein Occlusion, Disseminated Intravascular Coagulopathy, Branch Retinal Vein Occlusion, Hypertensive Fundus Changes, Ocular Ischemic Syndrome, Retinal Arterial Microaneurysms, Coat's Disease, Parafoveal Telangiectasis, Hemi-Retinal Vein Occlusion, Papillophlebitis, Central Retinal Artery Occlusion, Branch Retinal Artery Occlusion, Carotid Artery Disease (CAD), Frosted Branch Angitis, Sickle Cell Retinopathy and other Hemoglobinopathies, Angioid Streaks, Familial Exudative Vitreoretinopathy, Eales Disease.

TRAUMATIC/SURGICAL: Sympathetic Ophthalmia, Uveitic Retinal Disease, Retinal Detachment, Trauma, Laser, PDT, Photocoagulation, Hypoperfusion During Surgery, Radiation Retinopathy, Bone Marrow Transplant Retinopathy.

PROLIFERATIVE DISORDERS: Proliferative Vitreal Retinopathy and Epiretinal Membranes, Proliferative Diabetic Retinopathy.

INFECTIOUS DISORDERS: Ocular Histoplasmosis, Ocular Toxocariasis, Presumed Ocular Histoplasmosis Syndrome (POHS), Endophthalmitis, Toxoplasmosis, Retinal Diseases Associated with HIV Infection, Choroidal Disease Associated with HIV Infection, Uveitic Disease Associated with HIV Infection, Viral Retinitis, Acute Retinal Necrosis, Progressive Outer Retinal Necrosis, Fungal Retinal Diseases, Ocular Syphilis, Ocular Tuberculosis, Diffuse Unilateral Subacute Neuroretinitis, Myiasis.

GENETIC DISORDERS: Retinitis Pigmentosa, Systemic Disorders with Accosiated Retinal Dystrophies, Congenital Stationary Night Blindness, Cone Dystrophies, Stargardt's Disease and Fundus Flavimaculatus, Best's Disease, Pattern Dystrophy of the Retinal Pigmented Epithelium, X-Linked Retinoschisis, Sorsby's Fundus Dystrophy, Benign Concentric Maculopathy, Bietti's Crystalline Dystrophy, pseudoxanthoma elasticum.

RETINAL TEARS/HOLES: Retinal Detachment, Macular Hole, Giant Retinal Tear.

TUMORS: Retinal Disease Associated with Tumors, Congenital Hypertrophy of the RPE, Posterior Uveal Melanoma, Choroidal Hemangioma, Choroidal Osteoma, Choroidal Metastasis, Combined Hamartoma of the Retina and Retinal Pigmented Epithelium, Retinoblastoma, Vasoproliferative Tumors of the Ocular Fundus, Retinal Astrocytoma, Intraocular Lymphoid Tumors.

MISCELLANEOUS: Punctate Inner Choroidopathy, Acute Posterior Multifocal Placoid Pigment Epitheliopathy, Myopic Retinal Degeneration, Acute Retinal Pigment Epithelitis and the like.

Each and every feature described herein, and each and every combination of two or more of such features, is included within the scope of the present invention provided that the features included in such a combination are not mutually inconsistent.

1. A method for treating an eye, the method comprising the steps of:
providing a device comprising a hollow sleeve having an open proximal end, a distal end, a substantially uniform inner diameter and a substantially uniform outer diameter extending between the proximal end and the distal end, the hollow sleeve terminating at the proximal end;
placing a substantially bioerodible implant including a therapeutic component and sized for placement in an eye, in the hollow sleeve;
passing the distal end of the hollow sleeve through a surface of an eye;
introducing the implant into the eye at a location within the eye by passing the implant out of the distal end of the hollow sleeve; and
withdrawing the hollow sleeve from the eye.

2. The method of claim 1 wherein the step of introducing the implant includes advancing a plunger element through the hollow sleeve in a distal direction in order to urge the implant out of the distal end of the hollow sleeve.

3. The method of claim 1 wherein the eye is a mammalian eye.

4. The method of claim 1 wherein the eye is a non-human animal eye.

5. The method of claim 1 wherein the eye is an equine eye.

6. The method of claim 1 wherein the eye is a human eye.

7. The method of claim 1 wherein the step of introducing comprises introducing the implant into an anterior chamber of the eye.

8. The method of claim 1 wherein the step of introducing comprises introducing the implant into a posterior chamber of the eye.

9. The method of claim 1 wherein the step of introducing comprises introducing the implant into a structure of the eye selected from the group consisting of the retina, the vitreous, the cornea, and the scleral of the eye.

10. The method of claim 1 wherein the step of introducing comprises introducing the implant into a structure of the eye selected from the group consisting of the meningeal space, the optic nerve, and the intraoptic nerve.

11. The method of claim 1 wherein the therapeutic component comprises an anti-inflammatory agent.

12. The method of claim 1 wherein the therapeutic component comprises a steroid component.

13. The method of claim 1 wherein the therapeutic component comprises a steroid component selected from the group consisting of cortisone, dexamethasone, hydrocortisone, methylprednisolone, prednisolone, prednisone, triamcinolone, and mixtures thereof.

14. The method of claim 1 wherein the therapeutic component comprises dexamethasone.

15. The method of claim 1 wherein the implant further includes a polymeric component.

16. The method of claim 11 wherein the polymeric component is selected from the group of polymeric components consisting of lactic acid polymers, glycolic acid polymers, copolymers of lactic acid and glycolic acid, and combinations thereof.

17. The method of claim 1 effective in treating an inflammation-mediated condition of the eye.

18. The method of claim 17 wherein the inflamation mediated condition of the eye is selected from the group consisting of uveitis, macular edema, acute macular degeneration, retinal detachment, ocular tumors, fungal infections, viral infections, multifocal choroiditis, diabetic uveitis, proliferative vitreoretinopathy, sympathetic ophthalmia, Vogt Koyanagi Harada syndrome, histoplasmosis, uveal diffusion, and combinations thereof.

19. The method of claim 1 wherein the implant comprises a generally cylindrically shaped implant configured to fit within the hollow sleeve.

20. The method of claim 19 wherein the inner diameter of the hollow sleeve is greater than an outer diameter of the implant.

21. The method of claim 1 wherein the hollow sleeve is structured to accommodate the implant in a substantially frictionless manner in order to maintain the implant substantially intact while the implant passes through the hollow sleeve and into the eye.

22. The method of claim 1 wherein the hollow sleeve comprises a fluoroplastic material.

23. The method of claim 1 wherein the hollow sleeve includes a material selected from the group consisting of polytetrafluoroethylene, polyperfluoroalkoxyl, ethylene propylene, polyfluorinated ethylene propylene, and combinations thereof.

24. A method of treating an inflammation-mediated condition of an eye comprising the steps of:
providing a device including a hollow sleeve having a distal end;
placing an implant sized for placement in an eye, in the hollow sleeve, the implant being substantially non-flexible and including a therapeutic component and a polymeric component;
passing the distal end of the hollow sleeve through a surface of the eye and into the eye;
introducing the implant into a vitreous of the eye by passing the implant out of the distal end of the hollow sleeve; and
withdrawing the hollow sleeve from the eye.

25. The method of claim 24 wherein the step of introducing the implant includes advancing a plunger element through the hollow sleeve in a distal direction in order to urge the implant out of the distal end of the sleeve and into the vitreous.

26. The method of claim 24 wherein the therapeutic component comprises a steroid component.

27. The method of claim 26 wherein the steroid component is selected from the group consisting of cortisone, dexamethasone, hydrocortisone, methylprednisolone, prednisolone, prednisone, triamcinolone and combinations thereof.

28. The method of claim 26 wherein the steroid component comprises dexamethasone.

29. The method of claim 24 wherein the polymeric component is selected from the group consisting of lactic acid polymers, glycolic acid polymers, copolymers of lactic acid and glycolic acid, and combinations thereof.

30. The method of claim 24 wherein the implant is effective in treating an inflamation mediated condition of the eye selected from the group consisting of uveitis, macular edema, acute macular degeneration, retinal detachment, ocular tumors, fungal infections, viral infections, multifocal choroiditis, diabetic uveitis, proliferative vitreoretinopathy, sympathetic ophthalmia, Vogt Koyanagi Harada syndrome, histoplasmosis, uveal diffusion, and combinations thereof.

31. The method of claim 24 wherein the hollow sleeve comprises a fluoroplastic material.

32. An ocular research system comprising:
a plurality of hollow sleeves, wherein each sleeve is substantially identical to each other sleeve, is sized to be inserted into a vitreous of an eye, and includes a proximal end, a distal end, a substantially uniform inner diameter and a substantially uniform outer diameter extending between the proximal end and the distal end.

33. The system of claim 32 wherein the plurality of hollow sleeves are produced by cutting a substantially uniform tube at approximately equally spaced apart intervals.

34. The system of claim 32 further comprising a plurality of plunger elements, each plunger element being structured to be slidably received within one of the hollow sleeves.

35. The system of claim 32 further comprising a plurality of ocular implants, each ocular implant including a therapeutic component and a matrix component and being sized to be slidably received within one of the hollow sleeves.

36. The system of claim 32 wherein each sleeve is sized to be inserted into a vitreous of a rabbit eye.

37. The system of claim 32 wherein each of the hollow sleeves includes a beveled tip on the distal end of the hollow sleeve.

38. A device for delivering an ocular implant into an eye, the device consisting essentially of:
a hollow sleeve including an open proximal end, a distal end, a beveled tip distal to the distal end, a substantially uniform inner diameter and a substantially uniform outer diameter extending between the proximal end and the distal end, the device terminating at and not extending beyond the proximal end, and sized to receive an therapeutic implant; and
a plunger element structured to be slidably received within the hollow sleeve;
the device being structured to be directly manually manipulable when used to introduce a biodegradable implant into a vitreous of an eye.
